(19) United States
(12) Patent Application Publication
(10) Pub. No.: US 2015/0064200 A1
(43) Pub. Date: Mar. 5, 2015

(54) METHODS AND COMPOSITIONS FOR 6-PHOSPHOGLUCONATE DEHYDROGENASE (6-PGD) AS A TARGET FOR LUNG CANCER THERAPY

(71) Applicant: BETH ISRAEL DEACONESS MEDICAL CENTER, INC., Boston, MA (US)

(72) Inventors: Vikas P. Sukhatme, Newton, MA (US); Barden Chunkong Chang, Brookline, MA (US)

(73) Assignee: Beth Israel Deaconess Medical Center, Inc., Boston, MA (US)

(21) Appl. No.: 14/390,252

(22) PCT Filed: Apr. 4, 2013
(86) PCT No.: PCT/US13/35268

§ 371 Date: Oct. 2, 2014

Related U.S. Application Data
(60) Provisional application No. 61/620,130, filed on Apr. 4, 2012.

(51) Int. Cl. A61K 39/395; C12Q 1/32; A61K 45/06; A61K 31/7088

(52) U.S. Cl. CPC A61K 39/3955; A61K 31/7088; C12Q 1/32; A61K 45/06
USPC 424/158.1; 514/44 A; 435/26

(57) ABSTRACT

The present invention relates to methods, compositions, and diagnostic tests for treating and diagnosing a proliferative disease that result in dysregulation of 6-phosphogluconate dehydrogenase. In particular, the methods and compositions include treatment of gefitinib/erlotinib resistant proliferative diseases such as lung cancer using a 6-phosphogluconate dehydrogenase antagonist.

### CROSS-REFERENCE TO RELATED APPLICATIONS

This application claims benefit of U.S. Provisional Application No. 61/620,130, filed Apr. 4, 2012, which is hereby incorporated by reference in its entirety.

### BACKGROUND OF THE INVENTION

The present invention relates to methods and compositions for treating lung cancer that results in dysregulation of 6-phosphogluconate dehydrogenase.

Altered cellular metabolism is a hallmark of cancer. The features include enhanced glucose uptake and glycolysis and a decrease in mitochondrial oxidative phosphorylation. Tumors utilize glycolysis to generate adenosine triphosphates (ATPs) even under high oxygen tension, a phenomenon known as aerobic glycolysis or the Warburg effect. To enable such high glycolytic rates, many tumors increase glucose uptake up to 15 times compared to adjacent normal tissues.

The pentose phosphate pathway (PPP) is another branch of the cellular metabolism that is known to be hyperactive in cancer. Flux measurements have determined that the activity of this pathway can be elevated by almost eight fold in the breast cancer cell line, MCF7, compared to normal human mammary epithelial cells, 48R HMEC. The PPP consists of the oxidative and the non-oxidative branches. While the importance of the non-oxidative branch in tumor progression has been characterized at the steps catalyzed by transketolases, the potential beneficial role of the oxidative branch in cancer has received less attention.

The oxidative branch of the PPP is a major source of NADPH, which regulates cellular redox balance and provides reductive power or synthesis of biomolecules. Glucose-6-phosphate enters this pathway via its oxidation by the gateway enzyme glucose-6-phosphate dehydrogenase (G6PD). Expression knockdown of G6PD sensitizes cancer cells to oxidative stress induced by chemical agents or radiotherapy, however, other studies show that genetic deletion experiments of G6PD was not necessary in pentose phosphate synthesis or proliferation. 6-phosphogluconate dehydrogenase (6-PGD) is the third enzyme in the PPP that catalyzes the conversion of 6-phosphogluconate to ribulose-5-phosphate and uses NADP+ as a cofactor. New therapeutic approaches and methods are needed to treat or prevent drug resistant forms of lung cancer to provide beneficial alternatives.

### SUMMARY OF THE INVENTION

The invention features a method of treating a subject having a proliferative disease, the method including administering to the subject a 6-phosphogluconate dehydrogenase antagonist in an amount sufficient to treat the proliferative disease.

In one aspect, the invention also features a method of treating a subject having a proliferative disease, the method including administering to the subject a 6-phosphogluconate dehydrogenase antagonist in an amount sufficient to treat the proliferative disease, wherein the proliferative disease is resistant to gefitinib/erlotinib.

In any of the embodiments described herein, the proliferative disease can be selected from the group consisting of leukemia, brain cancer, bladder cancer, breast cancer, cervical cancer, colorectal cancer, endometrial cancer, esophageal cancer, head and neck cancer, liver cancer, lung cancer, lymphoma, ovarian cancer, pancreatic cancer, prostate cancer, renal cancer, skin cancer, stomach cancer, testis cancer, thyroid cancer, and urothelial cancer.

In particular embodiments, the proliferative disease can be lung cancer. In another embodiment, the lung cancer can be selected from a group consisting of non-small cell lung cancer, small-cell lung cancer, carcinoid, sarcoma, squamous cell cancer, adenocarcinoma, and large cell carcinoma. In some embodiments, the lung cancer can be resistant to gefitinib/erlotinib.

The invention also features a method of treating a subject having lung cancer, the method including: determining if the lung cancer is resistant to gefitinib/erlotinib, and administering to the subject determined to have a lung cancer resistant to gefitinib/erlotinib a 6-phosphogluconate dehydrogenase antagonist in an amount sufficient to treat the lung cancer.

In some embodiments, the method further includes administering the 6-phosphogluconate dehydrogenase antagonist with an anticancer agent. In other embodiments, the 6-PGD antagonist and the anticancer agent together are present in an amount sufficient to treat cancer. In other embodiments, the 6-PGD antagonist or the anticancer agent is present in an amount sufficient to treat cancer. In particular embodiments, the 6-PGD antagonist and the anticancer agent act synergistically.

In another aspect, the invention also features a method for identifying a compound for treating a proliferative disease, the method including contacting a cell with a candidate compound and measuring 6-phosphogluconate dehydrogenase activity, wherein the presence of a decreased level of 6-phosphogluconate dehydrogenase activity in the cell, as compared to a normal reference sample, identifies the compound as a treatment for a proliferative disease.

The invention also features a method for identifying a treatment for proliferative disease, the method including contacting a cell with gefitinib/erlotinib and a candidate compound and measuring 6-phosphogluconate dehydrogenase activity, wherein the presence of a decreased level of 6-phosphogluconate dehydrogenase activity in the cell, as compared to a normal reference sample, identifies the compound as a treatment for a proliferative disease resistant to gefitinib/erlotinib.

In some embodiments, the cell is derived from a patient with lung cancer. In particular embodiments, the cell is resistant to gefitinib/erlotinib.

In another aspect, the invention also features a method for diagnosing a subject as having, or having a predisposition to a proliferative disease, the method including: determining the level of 6-PGD activity in a sample from the subject, comparing the level of 6-PGD activity with a normal reference sample, wherein the presence of an increased level of 6-PGD activity, as compared to the normal reference sample, results in diagnosing the subject as having, or having a predisposition to, the proliferative disease and, administering to the subject a 6-PGD antagonist in an amount sufficient to treat the proliferative disease.

For any of the methods or compositions described herein, the 6-phosphogluconate dehydrogenase antagonist is an RNAi agent (e.g., any described herein), an anti-6-PGD antibody (e.g., any described herein), or a small molecule inhibitor (e.g., glucose 1,6-diphosphate).

The invention also features a composition including a 6-PGD antagonist and an anticancer agent (e.g. any described herein).

The invention also features a composition including two or more 6-PGD antagonists (e.g., any described herein). In particular embodiments, the two or more 6-PGD antagonists act synergistically.

For any of the methods or compositions described herein, the anticancer agent is one or more of a chemotherapeutic agent, an antiangiogenic agent, an immunomodulatory agent, or an agent for metabolic therapy (e.g., any described herein).

### DEFINITIONS

By “amount sufficient” of an agent is meant the amount of the agent sufficient to effect beneficial or desired result (e.g., treatment of a proliferative disease, e.g., lung cancer), and, as such, an amount sufficient of the formulation is an amount sufficient to achieve a reduction in the expression level and/or activity of the 6-PGD gene or protein, as compared to the response obtained without administration of the composition.

By “6-phosphogluconate dehydrogenase antagonist” is meant an agent or compound that decreases or reduces 6-PGD gene expression, protein expression, or activity (e.g., enzymatic activity), as defined herein, compared to a control (e.g., a decrease by 5%, 10%, 15%, 20%, 25%, 30%, 35%, 40%, 45%, 50%, 55%, 60%, 65%, 70%, 75%, 80%, 85%, 90%, or 95%, as compared to a control or a normal reference sample). 6-PGD antagonists can be identified and tested by any useful method (e.g., any described herein).

By “decreased level of activity” of 6-PGD is meant a decrease in 6-PGD gene expression, protein expression, or activity (e.g., enzymatic activity), as compared to a control from a normal cell or normal tissue (e.g., a decrease of at least 2-fold, e.g., from about 2-fold to about 150-fold, e.g., from 5-fold to 150-fold, from 5-fold to 100-fold, from 10-fold to 150-fold, from 10-fold to 100-fold, from 50-fold to 150-fold, from 50-fold to 100-fold, from 75-fold to 150-fold, or from 75-fold to 100-fold, as compared to a control or a normal reference sample). Decrease level of activity can be determined using any useful methods known in the art or described herein. For example, a decrease level of activity can be determined as a decrease in 6-PGD gene expression or decrease in 6-PGD protein concentration (e.g., as determined by PCR or gel electrophoresis), as compared to a control (e.g., a sample including normal cell or normal tissue from one or more healthy subjects) or a normal reference sample, as defined herein. In another example, a decrease level of activity can be determined as a decrease in 6-PGD enzymatic activity, such as by measuring decreased NADPH formation (e.g., from 3-fold to 4-fold decreased formation), increased NADP+/NADPH ratio (e.g., from 5-fold to 15-fold, e.g., about 9-fold, increased ratio), as compared to a control or a normal reference sample. Decreased level of activity can be measured directly (e.g., decreased 6-PGD gene expression or decreased 6-PGD enzymatic activity) or indirectly, such as by measuring levels of one or more metabolites associated with decreased 6-PGD activity (e.g., by measuring one or more increased levels of 6-phospho-D-gluconate, D-glucono-δ-lactone-6-phosphate, glucose-6-phosphate, and fructose-6-phosphate, e.g., from 2-fold to 4-fold, e.g., about 3-fold, increased levels), and by measuring one or more (e.g., decreased levels of fructose-1,6-bisphosphate, dihydroxyacetone phosphate, and glyceraldehyde-3-phosphate, e.g. from 50-fold to 150-fold, e.g., from 75-fold to 150-fold, e.g., about 90-fold, decreased levels), as compared to a control or a normal reference sample.

By “reference sample” is meant any sample, standard, standard curve, or level that is used for comparison purposes. A “normal reference sample” can be, for example, a prior sample taken from the same subject; a sample from a normal healthy subject; a sample from a subject not having a disease associated with increased activity of 6-PGD (e.g., lung cancer); a sample from a subject that is diagnosed with a propensity to develop a disease associated with increased activity of 6-PGD (e.g., cancer), but does not yet show symptoms of the disorder; a sample from a subject that has been treated for a disease associated with increased activity of 6-PGD (e.g., cancer); or a sample of purified 6-PGD at a known normal concentration.

By “RNAi agent” is meant any agent or compound that exerts a gene silencing effect by hybridizing a target nucleic acid. RNAi agents include any nucleic acid molecules that are capable of mediating sequence-specific RNAi (e.g., under stringent conditions), for example, a short interfering RNA (siRNA), double-stranded RNA (dsRNA), microRNA (miRNA), short hairpin RNA (shRNA), short interfering oligonucleotide, short interfering nucleic acid, short interfering modified oligonucleotide, short interfering nucleic acid, short interfering modified oligonucleotide, chemically-modified siRNA, post-transcriptional gene silencing RNA (ptgsRNA), and Dicer-substrate RNA (DsiRNA).

By “resistant to gefitinib/erlotinib” is meant a form of cancer that contains an EGFR activating mutation, such as L858R, and T790M (e.g., non-small cell lung cancer, small-cell lung cancer, carcinoid, sarcoma, squamous cell cancer, adenocarcinoma, and large cell carcinoma), making the cancer 10%, 20%, 30%, 40%, 50%, 60%, 70%, 80%, or 90% less sensitive to gefitinib and erlotinib inhibition.

By “proliferative disease” is meant a condition characterized by rapidly dividing cells resulting in uncontrolled growth of new tissue, parts, and/or surrounding cells.

### BRIEF DESCRIPTION OF THE DRAWINGS

FIG. 1 shows the expression of 6-PGD as analyzed by western blotting in seven commercially available human lung cancer samples and two normal lung samples. Overexpression of 6-PGD is represented as Δ fold increase compared to normal samples. The identity and pathological information of the tumors is tabulated to the right.

FIG. 2A shows a graph of cell proliferation in H1975 cells retrovirally transduced to inducibly express a control or 6-PGD specific shRNA (shRNA1, shRNA2, and shRNA3). Western blot analysis of 6-PGD expression confirms the effect of the 6-PGD specific shRNAs.

FIG. 2B shows a graph of cell proliferation upon knockdown of endogenous 6-PGD using 6-PGD shRNA1 and rescue by stable overexpression of mouse 6-PGD (pLNCX2-ms6PGD). Western blot analysis shows the detection of human 6-PGD by a monoclonal antibody and overexpressed mouse 6-PGD by a polyclonal antibody.

FIG. 3A-C are images of cells stained for senescence-associated beta galactosidase (SA-β-GAL) activity upon knockdown of 6-PGD by shRNA1, or shRNA2, and control shRNA.

FIG. 3D shows a quantitative analysis of SA-β-GAL activity in cells with 6-PGD knockdown using a chromogenic assay.

FIG. 3E shows a western blot analysis of p53 expression in cells with 6-PGD knockdown.

FIG. 4A shows a graph of cell proliferation in H1975 cells upon knockdown of G6PD by siRNA.

FIG. 4B is a western blot analysis of G6PD expression upon siRNA knockdown.

FIG. 5A shows a graph of cellular NADPH levels in H1975 cells treated with 6-PGD shRNA1, 6-PGD shRNA2, or control shRNA.

FIG. 5B shows a graph of oxygen consumption in H1975 cells treated with 6-PGD shRNA1, 6-PGD shRNA2, or control shRNA.

FIG. 6A is a series of graphs showing cell count in H1975 cells treated with 6-PGD shRNA1 and tested in proliferation assays in either 2 g/L fructose or glucose. Exogenously expressed mouse 6-PGD restored cell proliferation in cells treated with 6-PGD shRNA1 in glucose but not fructose. Western blot analysis shows the expression of human 6-PGD detected by a monoclonal antibody and overexpressed mouse 6-PGD detected by a polyclonal antibody.

FIG. 6B is a series of graphs showing β-GAL activity in cells treated with 6-PGD shRNA1 and tested in proliferation assays in either 2 g/L fructose or glucose.

FIG. 7 is a graph showing cell count in H1975 cells treated with 6-PGD shRNA or control shRNA, and rescue of cell proliferation by G6PD knockdown using siRNA. Western blot analysis confirms the expression of 6-PGD and G6PD upon shRNA and siRNA treatment, respectively.

FIG. 8 is a graph showing tumor growth in mice upon 6-PGD knockdown by shRNA1, or shRNA2, and control shRNA.

### DETAILED DESCRIPTION

We have discovered that expression knockdown of 6-phosphogluconate dehydrogenase (6-PGD), the third enzyme in the pentose phosphate pathway (PPP), significantly inhibits growth of a gefitinib-resistant lung cancer line H1975 through induction of cellular senescence. Furthermore, we discovered that glucose promotes cellular senescence in H1975 cells lacking 6-PGD, effectively turning an essential energy source that is highly preferred by cancer cells into a growth inhibitor. In particular, higher expression of 6-PGD is associated with various types of lung cancer cells, such as those for adenocarcinoma, adeno-squamous cell carcinoma, large cell carcinoma, small cell carcinoma, squamous cell carcinoma, and small cell carcinoma. Accordingly, the compositions and methods described herein can be useful for treating a proliferative disease (e.g., lung cancer), including those that have developed resistance to existing treatments such as gefitinib/erlotinib. Examples of compositions include a 6-PGD antagonist, a combination of two or more 6-PGD antagonists, or a combination of a 6-PGD antagonist and an anticancer agent. Examples of compositions and methods are described in detail below.

### Indications

The methods and compositions of the invention include administration of one or more 6-PGD antagonists (e.g., RNAi agents) to a subject having a proliferative disease (e.g., lung cancer and/or a cancer associate with increased activity of 6-PGD, e.g., an increased activity of 6-PGD of at least 2-fold, e.g., from about 2-fold to about 150-fold, e.g., from 5-fold to 150-fold, from 5-fold to 100-fold, from 10-fold to 150-fold, from 10-fold to 100-fold, from 50-fold to 150-fold, from 50-fold to 100-fold, from 75-fold to 150-fold, or from 75-fold to 100-fold, as compared to a control or a normal reference sample), or at risk of developing a proliferative disease (e.g., an increased risk of at least 10%, 20%, 30%, 40%, 50%, 60%, 70%, 80%, 90%, or 100%).

Any useful methods can be used to determine one or more diseases having an increased activity of 6-PGD. For example, the expression of 6-PGD can be determined in a sample obtained from a subject having a disease (e.g., by using western blotting, DNA expression is often detected by Southern blotting or polymerase chain reaction (PCR), and RNA expression is often detected by northern blotting, PCR, or RNAse protection assays), and an increase in 6-PGD activity, as described herein, indicates a disease that can be treated with a 6-PGD antagonist.

Exemplary proliferative diseases include non-solid cancers and solid cancers, such as leukemia (e.g., chronic myeloid leukemia, acute myeloid leukemia, acute lymphoblastic leukemia, and chronic lymphocytic leukemia), brain cancer (e.g., ependymoma, glioma, medulloblastoma, meningioma, teratoid rhabdoid tumor, and teratoma), bladder cancer (e.g., adenocarcinoma, sarcoma, small cell carcinoma, squamous cell carcinoma, and transitional cell carcinoma), breast cancer (e.g., breast ductal carcinoma), cervical cancer, colorectal cancer, endometrial cancer, esophageal cancer (e.g., adenocarcinoma and squamous cell carcinoma), head and neck cancer, liver cancer (e.g., hepatocellular carcinoma, cholangiocarcinoma, and hemangioendothelioma), lung cancer (e.g., non-small cell lung cancer, small-cell lung cancer, carcinoid, sarcoma, squamous cell cancer, adenocarcinoma, and large cell carcinoma), lymphoma (e.g., malignant lymphoma), ovarian cancer (e.g., ovarian epithelial carcinoma and teratoma), pancreatic cancer, prostate cancer (e.g., adenocarcinoma and prostatic intraepithelial neoplasia), renal cancer, skin cancer (e.g., basal cell carcinoma, squamous cell carcinoma, and malignant melanoma), stomach cancer, testis cancer, thyroid cancer, and urothelial cancer. In a preferred embodiment, the disease is lung cancer.

The methods and compositions described herein can also be used to treat cancers having one or more particular mutations that confer resistance to first-line anticancer agents. Exemplary cancers having mutations include non-small cell lung cancer having a T790M or a L747S mutation in EGFR kinase, a somatic activating mutation in the tyrosine-kinase pocket of EGFR (e.g., a deletion in exon 19 or a substitution in exon 21, e.g., L858R), or a mutation present in tyrosine kinase inhibitor-resistant cell line H1975; and brain cancer, breast cancer, colorectal cancer, lung cancer, and stomach cancer having a E542K, E545K, H1047R, P539R, or H1047L mutation in the PIK3CA gene (encoding a p110α of class IA of PI3K) (e.g., lung cancer having a H1047R mutation in PIK3CA).

### Diagnostic Methods

Increased activity of 6-PGD can also be used for the diagnosis of a proliferative disease, such as cancer (e.g., non-small cell lung cancer, small-cell lung cancer, carcinoid, sarcoma, squamous cell cancer, adenocarcinoma, and large cell carcinoma), or a risk of developing such a disease.

A subject having a disease associated with increased activity of 6-PGD (e.g., a proliferative disease, or a propensity to develop such as disease, will show an alteration, e.g., an increase or a decrease of 10%, 20%, 30%, 40%, 50%, 60%, 70%, 80%, 90%, or more) in the expression or biological activity of one or more activated or downregulated enzymes and analytes of 6-PGD (e.g., glucose 6-phosphate dehydrogenase, 6-phosphogluconolactonase, ribulose 5-phosphate isomerase, ribulose 5-phosphate 3-epimerase, transketolase, transaldolase, NADP+, NADPH, NADP⁺/NADPH ratio, ROS, oxygen levels, and dNTP/NTP levels) or in a cellular parameter (e.g., apoptosis, senescence, and proliferation).

In one example, an increase in 6-PGD gene or protein expression or 6-PGD enzymatic activity, as compared to a normal reference sample or control, is indicative of cancer, e.g., lung cancer, or a risk of developing the same.

In another example, an increase in 6-PGD gene or protein expression or 6-PGD enzymatic activity is determined indirectly, such as by measuring one or more of increased NADPH formation (e.g., from 3-fold to 4-fold increased formation), decreased NADP+/NADPH ratio (e.g., from 5-fold to 15-fold, e.g., about 9-fold, decreased ratio), as compared to a control or a normal reference sample.

Standard methods may be used to measure analyte levels or cellular parameters in any bodily fluid, including, but not limited to, urine, blood, serum, plasma, saliva, or cerebrospinal fluid. Such methods include immunoassay, ELISA, Western blotting using antibodies directed to 6-PGD and quantitative enzyme immunoassay techniques. ELISA assays are the preferred method for measuring polypeptide levels. Accordingly, the measurement of antibodies specific to 6-PGD in a subject may also be used for the diagnosis of cancer, e.g., lung cancer, or a risk of developing the same.

In one embodiment, a subject having a proliferative disease (e.g., lung cancer, or a risk of developing the same), will show an increase in the expression of a nucleic acid encoding 6-PGD. Methods for detecting such alterations are standard in the art. In one example Northern blotting or real-time PCR is used to detect mRNA levels.

In another embodiment, hybridization at high stringency with PCR probes that are capable of detecting 6-PGD, including genomic sequences, or closely related molecules, may be used to hybridize to a nucleic acid sequence derived from a subject having disease associated with increased activity of 6-PGD, e.g., cancer. The specificity of the probe, whether it is made from a highly specific region, e.g., the 5′-regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences. Hybridization techniques may be used to identify mutations in a nucleic acid molecule, or may be used to monitor expression levels of a gene encoding a polypeptide of the invention.

Diagnostic methods can include measurement of absolute levels of a polypeptide, or nucleic acid. In any of the diagnostic methods, the level of a polypeptide or nucleic acid, or any combination thereof, can be measured at least two different times from the same subject and an alteration in the levels (e.g., by 10%, 20%, 30%, 40%, 50%, 60%, 70%, 80%, 90%, or more) over time is used as an indicator of a disease associated with increased activity of 6-PGD, e.g., cancer, or the propensity to develop the same. It will be understood by the skilled artisan that for diagnostic methods that include comparing of the polypeptide, or nucleic acid to a reference level, particularly a prior sample taken from the same subject, a change over time with respect to the baseline level can be used as a diagnostic indicator of a cancer, or a predisposition to develop the same. The diagnostic methods described herein can be used individually or in combination with any other diagnostic method described herein for a more accurate diagnosis of the presence of severity of, or predisposition to a disease associated with increased activity of 6-PGD (e.g., cancer, or a predisposition to the same).

### 6-PGD Antagonists

6-PGD antagonists include one or more agents or compounds that directly or indirectly inhibit 6-PGD gene expression, protein expression, or enzymatic activity. Exemplary 6-PGD antagonists include an RNAi agent, (e.g., a shRNA for 6-PGD, as described herein), an anti-6-PGD antibody, and small molecule inhibitors. Additional 6-PGD antagonists can be identified by any useful method, such as by inhibiting or activating one or more proteins upstream of 6-PGD in the PPP that results in 6-PGD inhibition.

### RNAi Agents

6-PGD inhibitors include one or more RNAi agents that inhibit 6-PGD gene expression in a cell in vitro or in vivo (e.g., in a subject). The RNAi agents can include different types of double-stranded molecules that include either RNA:RNA or RNA:DNA strands. These agents can be introduced to cells in a variety of structures, including a duplex (e.g., with or without overhangs on the 3′-terminus), a hairpin loop, or an expression vector that express one or more polynucleotides capable of forming a double-stranded polynucleotide alone or in combination with another polynucleotide.

Exemplary RNAi agents include siRNA, shRNA, DsiRNA, and miRNA agents. Generally these agents are about 10 to about 40 nucleotides in length, and preferred lengths for particular RNAi agents include siRNA that are double-stranded RNA molecules of 16 to 30 nucleotides in length (e.g., 18 to 25 nucleotides, e.g., 21 nucleotides); shRNA that are single-stranded RNA molecules in which a hairpin loop structure is present and a stem length is between 19 to 29 nucleotides in length (e.g., 19 to 21 nucleotides or 25 to 29 nucleotides) or a loop size is between 4 to 23 nucleotides in length; DsiRNA that are double-stranded RNA agents of 25 to 35 nucleotides in length; and miRNA that are single-stranded RNA molecules of 17 to 25 nucleotides (e.g., 21 to 23 nucleotides) in length.

The RNAi agent can have any useful nucleic acid sequence, including a nucleic acid sequence having one or more DNA molecules, RNA molecules, or modified forms (e.g., a modified backbone composition or 2′-deoxy, or 2′-O-methyl modifications) or combinations thereof. Additionally, the RNAi agent can contain 5′- and/or 3′-terminal modifications and include blunt and overhanging nucleotides at these teimini, or combinations thereof. Exemplary modifications include a 5′-dideoxythymidine overhang, such as for siRNAi; a 3′-UU or 3′-dTdT overhang, such as for shRNA; one or more G-U mismatches between the two strands of the shRNA stem; or a single-stranded nucleotide overhang at the 3′-terminal of the antisense or sense strand of 1 to 4 nucleotides (e.g., 1 or 2 nucleotides) for DsiRNA.

Methods of producing antisense and sense nucleotides, as well as corresponding duplexes or hairpin loops, are known in the art and can be readily adapted to produce an antisense oligonucleotide that targets any target nucleic acid sequence. RNAi agents include at least one antisense nucleotide sequence that is directed to a target nucleic acid (e.g., a target gene, e.g., a 6-PGD gene). Antisense nucleotides are single strands of DNA or RNA that are complementary to a chosen target sequence. In the case of antisense RNA, they prevent translation of complementary RNA strands by binding to it. Antisense DNA can be used to target an antisense nucleotides contain from about 10 to about 40 nucleotides, more preferably about 15 to about 30 nucleotides. The antisense nucleotide can have up to 80%, 85%, 90%, 95%, 99%, or even 100% complementary to the desired target gene.

6-PGD antagonists include one or more RNAi agents. Exemplary RNAi agents include shRNA agents such as those provided in Table 1 from Open Biosystems (Lafayette, Colo.), siRNA agents such as those provided in Table 2 from Invitrogen I (Carlsbad, Calif.), and candidate siRNAs, determined by any siRNA prediction tools known in the art (e.g., Human siRNA Database (HuSiDa), provided in Table 3, where sequences are provided in the 5′ to 3′ direction, unless otherwise specified. RNAi agents also include commercially available agents, such as those available from OriGene Technologies (Rockville, Md.) and Santa Cruz Biotechnologies, Inc. (Santa Cruz, Calif.).

TABLE 1

| shRNA | Oligo ID | Mature Sequence |
|---|---|---|
| #1 | V3LHS_381993 | AGU UUG AUG GUG AUA AGA A |
| #2 | V3LHS_381995 | AGG ACU GUC UCC AAA GUU G |
| #3 | V3LHS_381996 | CGG AAC UUC AGA ACC UCC U |

TABLE 2

| siRNA | Primer Name | Sequence |
|---|---|---|
| #1 | PGDHSS107894 | 5′-GCC ACU UCG UGA AGA |
| | | UGG UGC ACA A-3′ |
| #2 | PGDHSS107895 | 5′-GCC CAG GCC UUU GAG |
| | | GAU UGG AAU A-3′ |
| #3 | PGDHSS182268 | 5′-CAG AAC CUC CUA CUG |
| | | GAC GAC UUC U-3′ |

TABLE 3

| | |
|---|---|
| Gene name: PGD | Organism: Homo sapiens |
| Accession: NM_002631 | GI Number: 40068517 |
| Total Length: 1937 | ORF Region 91 to 1542 |

Description: Homo sapiens phosphogluconate dehydrogenase (PGD), mRNA.
Targeted Transcripts: NM_002631
# Candidates: 50

| | | | | | Low | Requires |
|---|---|---|---|---|---|---|
| | | Start | GC | | Seed | Sense |
| Sense Strand Sequence | Region | position | % | Score | Freq? | Mods? |
| AGUCAGUGGUGGAGAGGAA | ORF | 471 | 53 | 94 | No | No |
| CAUCAUUGACGGAGGAAAU | ORF | 381 | 42 | 91 | No | No |
| GGAAUAAGACAGAGCUAGA | ORF | 758 | 42 | 91 | No | No |
| GCACAACGGGAUAGAGUAU | ORF | 648 | 47 | 89 | No | No |
| CCUAUGAACUCUUGGCCAA | ORF | 1451 | 47 | 89 | No | No |
| AGACAGAGCUAGACUCAUU | ORF | 764 | 42 | 87 | No | No |
| UCGUGAAGAUGGUGCACAA | ORF | 635 | 47 | 86 | No | No |
| GAAUAUAGGGACACCACAA | ORF | 403 | 42 | 85 | No | No |
| CUGCCAAAGAUCAGGGACA | ORF | 841 | 53 | 83 | No | Yes |
| CAUUAGAAGUGUAUUCCUA | ORF | 1191 | 32 | 83 | No | No |
| CCAAUGAGGCAAAGGGAAC | ORF | 221 | 53 | 82 | No | No |
| GCCGAGACCUCAAGGCCAA | ORF | 428 | 63 | 81 | No | No |
| GCUCAUGCCAGGAGGGAAC | ORF | 510 | 63 | 81 | No | No |
| GCAGAAGGGCACAGGGAAG | ORF | 867 | 63 | 81 | No | No |
| CCUUUGAGGAUUGGAAUAA | ORF | 746 | 37 | 80 | No | No |
| CCUCAAGGCCAAGGGAAUU | ORF | 435 | 53 | 79 | No | No |
| ACUCAUUCCUGAUUGAAAU | ORE | 776 | 32 | 79 | No | No |
| GAAAGAUAAAGGAUGCAUU | ORF | 1211 | 32 | 79 | No | No |
| AGGAUGCAUUUGAUCGAAA | ORF | 1220 | 37 | 79 | Yes | No |
| ACCUCAAGGCCAAGGGAAU | ORF | 434 | 53 | 78 | No | No |
| GCCUUUGAGGAUUGGAAUA | ORF | 745 | 42 | 78 | No | No |
| CUGCAUCAUUAGAAGUGUA | ORF | 1185 | 37 | 78 | No | No |
| AGAUAAAGGAUGCAUUUGA | ORF | 1214 | 32 | 77 | No | Yes |
| UAAUAGGACUGUCUCCAAA | ORF | 186 | 37 | 76 | No | Yes |
| GCCCAGUCCCUGAAAGAGA | ORF | 253 | 58 | 76 | No | No |
| UUGGAAUAAGACAGAGCUA | ORF | 756 | 37 | 76 | No | Yes |
| GAUAAAGGAUGCAUUUGAU | ORF | 1215 | 32 | 76 | No | No |
| AGGAUUGGAAUAAGACAGA | ORF | 752 | 37 | 75 | No | Yes |
| GAGCAGGCCACUUCGUGAA | ORF | 623 | 58 | 74 | Yes | No |
| GAUGGUGCACAACGGGAUA | ORF | 642 | 53 | 74 | Yes | No |
| GUGCACAACGGGAUAGAGU | ORF | 646 | 53 | 74 | No | Yes |
| GGGAUAGAGUAUGGGGACA | ORF | 655 | 53 | 74 | No | No |
| CCAAUAUUCUCAAGUUCCA | ORF | 800 | 37 | 74 | No | No |
| GAUCAUCUCUUACGCUCAA | ORF | 1083 | 42 | 74 | Yes | No |
| GCUUUAUGCUGCUAAGGCA | ORF | 1103 | 47 | 74 | No | No |
| CAUCAUUAGAAGUGUAUUC | ORF | 1188 | 32 | 74 | No | Yes |
| CCUAGGAAAGAUAAAGGAU | ORF | 1206 | 37 | 74 | No | No |
| GUGCCCAGUCCCUGAAAGA | ORF | 251 | 58 | 73 | No | Yes |
| GGAUAGAGUAUGGGGACAU | ORF | 656 | 47 | 73 | No | No |
| GCAGCUGAUCUGUGAGGCA | ORF | 675 | 58 | 73 | No | No |
| GGCAGAAGGGCACAGGGAA | ORF | 866 | 63 | 73 | No | No |
| GCAGGCAGCCACCGAGUUU | ORF | 1119 | 63 | 73 | Yes | No |
| CAUGAAUGACCACGGCUUU | ORF | 153 | 47 | 73 | Yes | No |
| CAUCAUCAUUGACGGAGGA | ORF | 378 | 47 | 72 | No | Yes |
| AGACCAUCUUCCAAGGCAU | ORF | 551 | 47 | 72 | No | No |
| CAGCCAAUAUUCUCAAGUU | ORF | 797 | 37 | 72 | No | No |
| UCUCAAUUAUGGUGGCAUC | ORF | 1146 | 42 | 72 | No | Yes |
| UAGGAAAGAUAAAGGAUGC | ORF | 1208 | 37 | 72 | No | Yes |
| GGUACAGACAUGAGAUGCU | ORF | 1385 | 47 | 72 | No | No |
| GAGAUGAGGGAGCAGGCCA | ORF | 614 | 63 | 71 | No | Yes |

### Other 6-PGD Inhibitors

6-PGD antagonists also include one or more anti-6-PGD antibodies. Exemplary antibodies include those listed in Table 4. Antibodies are also commercially available from Abeam (Cambridge, Mass.), Atlast Antibodies AB (Stockholm, Sweden, Novus Biologicals (Littleton, Colo.), LifeSpan Biosciences (Seattle, Wash.), and Santa Cruz Biotechnology (Santa Cruz, Calif.).

TABLE 4

| 6-PGD Antibodies | Company Name | Catalog Number |
|---|---|---|
| #1 | Santa Cruz Biotechnology | sc-100316 |
| #2 | Sigma | S AB1100532 |
| #3 | Santa Cruz Biotechnology | sc-138521 |
| #4 | Santa Cruz Biotechnology | sc-138520 |
| #5 | Santa Cruz Biotechnology | sc-138519 |
| #6 | Sigma | WH0005226M1 |
| #7 | Sigma | SAB1100531 |
| #8 | Sigma | HPA031314 |
| #9 | Sigma | SAB1410960 |
| #10 | Sigma | SAB1406252 |
| #11 | Abcam | ab129199 |
| #12 | Abcam | ab55614 |
| #13 | Abcam | ab125863 |
| #14 | Abcam | ab96225 |
| #15 | Origene | TA308104 |
| #16 | Novus Biologicals | H00005226-M01 |
| #17 | Novus Biologicals | NBP1-85925 |
| #18 | Novus Biologicals | H00005226-D01P |
| #19 | Novus Biologicals | NBP1-31589 |
| #20 | Novus Biologicals | NBP1-41231 |

6-PGD small molecule inhibitors include one or more compounds that inhibit one or more proteins upstream of 6-PGD in the PPP, such as a G6PD inhibitor, a 6-phosphogluconolactonase; one or more compounds that inhibit one or more proteins downstream of 6-PGD in the PPP, such as a transketolase inhibitor, a transaldolase inhibitor, a ribulose 5-phosphate isomerase inhibitor, a ribulose 5-phosphate 3-epimerase inhibitor, and dual inhibitors of any of these proteins (e.g., a dual G6PD/transketolase inhibitor, or a dual 6-phosphogluconolactonase/transaldolase inhibitor). An exemplary 6-PGD small molecule inhibitor further includes glucose 1,6-diphosphate.

### Combination Therapy

The methods and compositions include combinations of a 6-PGD antagonist and a therapeutic agent, such as an anticancer agent. Exemplary anticancer agents include chemotherapeutic agents (e.g., arsenic trioxide, cisplatin, carboplatin, chlorambucil, melphalan, nedaplatin, oxaliplatin, triplatin tetranitrate, satraplatin, imatinib, nilotinib, dasatinib, and radicicol), immunomodulatory agents (e.g., methotrexate, leflunomide, cyclophosphamide, cyclosporine A, minocycline, azathioprine, antibiotics (e.g., tacrolimus), methylprednisolone, corticosteroids, steroids, mycophenolate mofetil, rapamycin, mizoribine, deoxyspergualin, brequinar, T cell receptor modulators, and cytokine receptor modulators), antiangiogenic agents (e.g., bevacizumab, suramin, and etrathiomolybdate), mitotic inhibitors (e.g., paclitaxel, vinorelbine, docetaxel, abazitaxel, ixabepilone, larotaxel, ortataxel, tesetaxel, vinblastine, vincristine, vinflunine, and vindesine), nucleoside analogs (e.g., gemcitabine, azacitidine, capecitabine, carmofur, cladribine, clofarabine, cytarabine, decitabine, floxuridine, fludarabine, fluorouracil, mercaptopurine, pentostatin, tegafur, and thioguanine), DNA intercalating agents (e.g., doxorubicin, actinomycin, bleomycin, mitomycin, and plicamycin), topoisomerase inhibitors (e.g., irinotecan, aclarubicin, amrubicin, belotecan, camptothecin, daunorubicin, epirubicin, etoposide, idarubicin, mitoxantrone, pirarubicin, pixantrone, rubitecan, teniposide, topotecan, valrubicin, and zorubicin), folate antimetabolites (e.g., pemetrexed, aminopterin, methotrexate, pralatrexate, and raltitrexed), and other targeting agents (e.g., agents that target particular enzymes in a metabolic pathway or proteins involved in cancer or agents that target particular organs or types of cancers), and combinations thereof.

In particular embodiments, the combination include a 6-PGD antagonist and an anticancer agent targeting the glycolytic metabolism of tumors. Exemplary combinations of 6-PGD antagonists include the combination of an RNAi agent (e.g., any described herein, such as those in Tables 1-3) and an anticancer agent, such as an RNAi agent and a HK2 inhibitor (e.g., phloretin, silybin/silibinin, 2DG, lonidamine, and 3-bromopyruvate); an RNAi agent and a PI3K inhibitor (e.g., wortmannin, demethoxyviridin, LY294002, quercetin, myricetin, staurosporine, GDC-0941, NVP-BEZ235, ZSTK474, PX-866, and XL-147, e.g., wortmannin, e.g., LY294002); an RNAi agent and an RTK inhibitor (e.g., erlotinib, gefitinib, vandetanib, afatinib, axitinib, cediranib, cetuximab, lapatinib, lestaurtinib, neratinib, panitumumab, pazopanib, regorafenib, semaxanib, sorafenib, sunitinib, toceranib, and trastuzumab, e.g., erlotinib, e.g., gefitinib); an RNAi agent and a LDH5 inhibitor (e.g., shikonin, alkannin, AT-101, and FX11); an RNAi agent and an AMPK activator (e.g., metformin); an RNAi agent and a CA9 inhibitor (e.g., indisulam); an RNAi agent and a HIF-1 inhibitor (e.g., BAY87-2243, EZN-2968, and quarfloxin); and an RNAi agent and a dual PFK2/PFKFB3 dual inhibitor (e.g., 3PO).

Another exemplary combination includes an anti-6-PGD antibody (e.g., any described herein, such as those in Table 4) and another 6-PGD antagonist, such as the combination of an anti-6-PGD antibody and an RNAi agent (e.g., any described herein, such as those in Table 1-3). Yet another exemplary combination includes an anti-6-PGD antibody and an anticancer agent targeting the glycolytic metabolism of tumors, such as, an anti-6-PGD antibody and a HK2 inhibitor (e.g., phloretin, silybin/silibinin, 2DG, lonidamine, and 3-bromopyruvate); an anti-6-PGD antibody and a PI3K inhibitor (e.g., wortmannin, demethoxyviridin, LY294002, quercetin, myricetin, staurosporine, GDC-0941, NVP-BEZ235, ZSTK474, PX-866, and XL-147, e.g., wortmannin, e.g., LY294002); an anti-6-PGD antibody and an RTK inhibitor (e.g., erlotinib, gefitinib, vandetanib, afatinib, axitinib, cediranib, cetuximab, lapatinib, lestaurtinib, neratinib, panitumumab, pazopanib, regorafenib, semaxanib, sorafenib, sunitinib, toceranib, and trastuzumab, e.g., erlotinib, e.g., gefitinib); an anti-6-PGD antibody and a LDH5 inhibitor (e.g., shikonin, alkannin, AT-101, and FX11); an anti-6-PGD antibody and an AMPK activator (e.g., metformin); an anti-6-PGD antibody and a CA9 inhibitor (e.g., indisulam); an anti-6-PGD antibody and a HIF-1 inhibitor (e.g., BAY87-2243, EZN-2968, and quarfloxin); and an anti-6-PGD antibody and a dual PFK2/PFKFB3 dual inhibitor (e.g., 3PO).

### Administration and Dosage

The present invention also relates to pharmaceutical compositions that contain one or more 6-PGD antagonists or a combination of a 6-PGD antagonist and a therapeutic agent (e.g., a combination of a 6-PGD antagonist and an anticancer agent). The composition can be formulated for use in a variety of drug delivery systems. One or more physiologically acceptable excipients or carriers can also be included in the composition for proper formulation. Suitable formulations for use in the present invention are known in the art.

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration

The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a patient with a clinically determined predisposition or increased susceptibility to development of a tumor or cancer. Compositions of the invention can be administered to the patient (e.g., a human) in an amount sufficient to delay, reduce, or preferably prevent the onset of clinical disease or tumorigenesis. In therapeutic applications, compositions are administered to a patient (e.g., a human) already suffering from a proliferative disease in an amount sufficient to cure or at least partially arrest the symptoms of the condition and its complications. For example, in the treatment of a proliferative disease, an agent or compound which decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

Amounts effective for this use may depend on the severity of the disease or condition and the weight and general state of the patient, but generally range from about 0.5 mg to about 3000 mg of the agent or agents per dose per patient. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more agents present within the compositions of the invention and used in the methods of this invention applied to mammals (e.g., humans) can be determined by the ordinarily-skilled artisan with consideration of individual differences in age, weight, and the condition of the mammal. The agents of the invention are administered to a subject (e.g. a mammal, such as a human) in an effective amount, which is an amount that produces a desirable result in a treated subject (e.g. the slowing or remission of a cancer or neurodegenerative disorder). Such therapeutically effective amounts can be determined empirically by those of skill in the art.

The patient may also receive an agent in the range of about 0.1 to 3,000 mg per dose one or more times per week (e.g., 2, 3, 4, 5, 6, or 7 or more times per week), 0.1 to 2,500 (e.g., 2,000, 1,500, 1,000, 500, 100, 10, 1, 0.5, or 0.1) mg dose per week. A patient may also receive an agent of the composition in the range of 0.1 to 3,000 mg per dose once every two or three weeks.

Single or multiple administrations of the compositions of the invention comprising an effective amount can be carried out with dose levels and pattern being selected by the treating physician. The dose and administration schedule can be determined and adjusted based on the severity of the disease or condition in the patient, which may be monitored throughout the course of treatment according to the methods commonly practiced by clinicians or those described herein.

The compounds and formulations of the present invention may be used in combination with either conventional methods of treatment or therapy or may be used separately from conventional methods of treatment or therapy. When the compounds and formulations of this invention are administered in combination therapies with other agents, they may be administered sequentially or concurrently to an individual. Alternatively, pharmaceutical compositions according to the present invention include a combination of a compound or formulation of the present invention in association with a pharmaceutically acceptable excipient, as described herein, and another therapeutic or prophylactic agent known in the art.

The formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Screening Assays to Identify One or More 6-PGD Antagonists

As discussed above, we have discovered that increased activity of 6-PGD is correlated with cancer. Based on these discoveries, 6-PGD and proteins upstream or downstream of 6-PGD in the PPP (e.g., including but not limited to glucose 6-phosphate dehydrogenase, 6-phosphogluconolactonase, ribulose 5-phosphate isomerase, ribulose 5-phosphate 3-epimerase, transketolase, and transaldolase), as well as other analytes or cellular parameters related to increased activity of 6-PGD (e.g., NADP+, NADPH, NADP⁺/NADPH ratio, ROS, oxygen levels, and dNTP/NTP levels) are useful for the high-throughput low-cost screening of candidate compounds to identify those that modulate, alter, or decrease (e.g., by at least 10%, 20%, 30%, 40%, 50%, 60%, 70%, 80%, 90%, 95%, or more) the expression or biological activity of 6-PGD. Compounds that decrease the expression or biological activity of 6-PGD or modulate, alter, decrease, or increase expression or biological activity of proteins upstream and downstream of 6-PGD (e.g., glucose 6-phosphate dehydrogenase, 6-phosphogluconolactonase, ribulose 5-phosphate isomerase, ribulose 5-phosphate 3-epimerase, transketolase, and transaldolase) can be used for the treatment or prevention of a disease associated with increased activity of 6-PGD, e.g., cancer, or a propensity to develop such as disease.

In particular examples, candidate compounds having one or more of the following properties are considered 6-PGD inhibitors: decreased NADPH formation (e.g., from 3-fold to 4-fold decreased formation), increased NADP⁺/NADPH ratio (e.g., from 1.5-fold to 3-fold, e.g., about 2-fold, increased ratio), increased ROS activity, increased glutathione levels (e.g., from 2-fold to 4-fold, e.g., about 3-fold, increased levels), decreased ribose-5-phosphate (R5P) levels (e.g., from 2-fold to 4-fold, e.g., about 3-fold, decreased levels) decreased erythrose-4-phosphate (E4P) levels (e.g., from 2-fold to 4-fold, e.g., about 3-fold, decreased levels), increased apoptosis, decreased cellular proliferation, increased senescence-associated β-GAL activity, or increased p53 expression, as compared to a control or a normal reference sample (e.g., a sample from a subject that has been treated for a disease associated with increased activity of 6-PGD, e.g., cancer; or a sample of purified 6-PGD at a known normal concentration). Candidate compounds can be tested for their effect on 6-PGD activity using assays known in the art.

Candidate compounds can also be tested for their effect on 6-PGD activity using any particular cell based assays described herein. Standard methods may be used to measure analyte levels or cellular parameters in any bodily fluid, including, but not limited to, urine, blood, serum, plasma, saliva, or cerebrospinal fluid. Such methods include immunoassay, ELISA, Western blotting using antibodies directed to 6-PGD and quantitative enzyme immunoassay techniques. ELISA assays are the preferred method for measuring polypeptide levels. Accordingly, the measurement of antibodies specific to 6-PGD in a subject may also be used to determine if a compound has effects on 6-PGD activity.

In one embodiment, a compound that affects 6-PGD activity may show a decrease in the expression of a nucleic acid encoding 6-PGD. Methods for detecting such alterations are standard in the art. In one example Northern blotting or real-time PCR is used to detect mRNA levels.

In another embodiment, hybridization techniques may be used to monitor expression levels of a gene encoding a polypeptide of the invention upon treatment with a candidate compound.

In a further embodiment, a reporter gene such as a gene encoding GFP or luciferase can be fused to the 6-PGD promoter to monitor the expression levels of 6-PGD upon treatment with a candidate compound.

In general, candidate compounds are identified from large libraries of both natural product or synthetic (or semi-synthetic) extracts, chemical libraries, or from polypeptide or nucleic acid libraries, according to methods known in the art. Those skilled in the field of drug discovery and development will understand that the precise source of test extracts or compounds is not critical to the screening procedure(s) of the invention.

### EXAMPLES

### Example 1

### 6-PGD is Upregulated in Lung Cancer

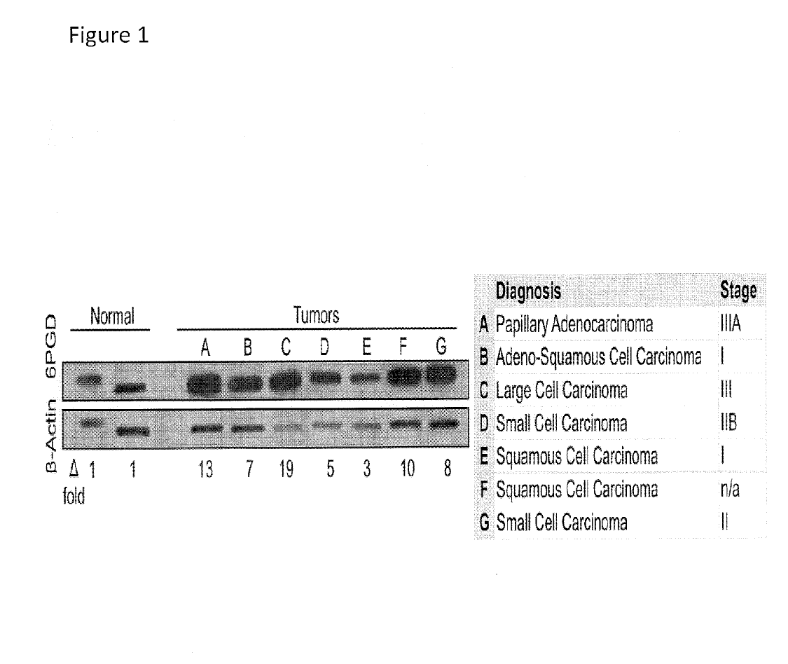

To examine the potential role of 6-PGD in lung cancer, we measured the expression level of the protein in multiple commercially available human lung cancer samples against normal lung tissue by western blot analysis. As shown in FIG. 1, 6-PGD expression was upregulated in all seven lung cancer samples examined when compared to two normal adult lung tissues. Therefore, 6-PGD may play an important role in lung cancer biology, and lung cancer cells may be more sensitive to 6-PGD inhibition compared to normal lung tissues.

### Example 2

### Expression Knockdown of 6-PGD Inhibits Proliferation of NSCLC In Vitro

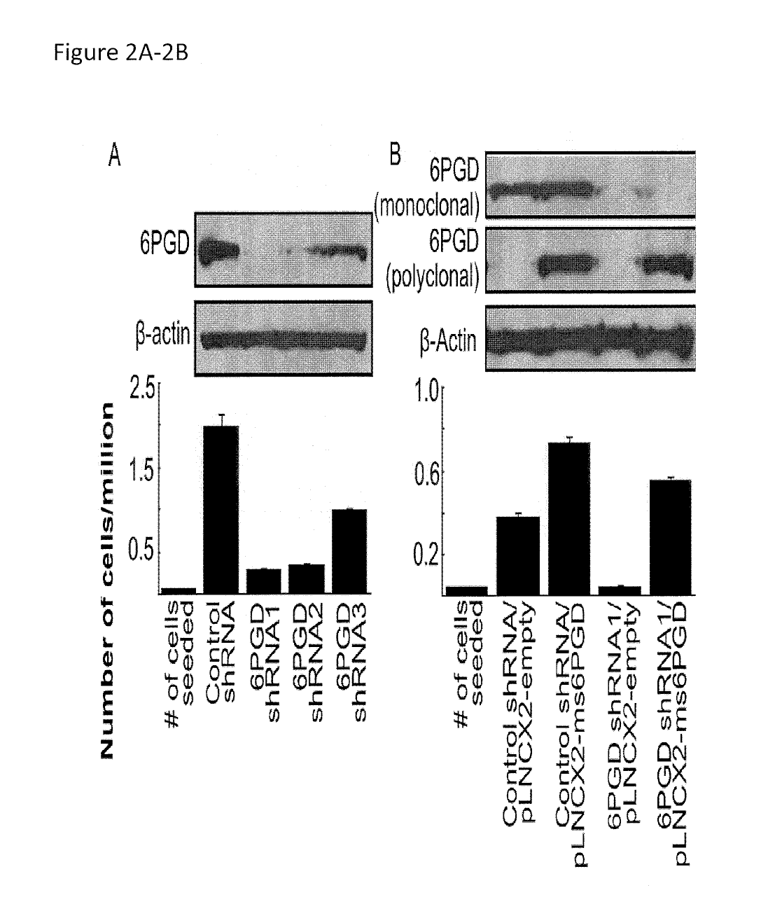

To test the functional role of 6-PGD in human non small cell lung carcinoma (NSCLC), we used H1975 cells as our model system (FIG. 2A). These adenocarcinoma cells harbor two mutations in the EGFR (L858R/T790M) and are widely used to study the mechanism and treatment of gefitinib/erlotinib-resistant lung cancer cells. We chose to use an inducible shRNA system to knock down 6-PGD expression. Three nonoverlapping shRNAs were chosen in these experiments. All three shRNAs significantly reduced the expression of 6-PGD, with shRNA1 and 2 being the most effective. Importantly, 6-PGD expression knockdown caused a substantial decrease in cell proliferation. To further corroborate the involvement of 6-PGD in NSCLC proliferation in vitro, we performed expression rescue experiments (FIG. 2B). H1975 cells expressing control shRNA or 6-PGD shRNA1 were transduced by either empty- or mouse-6PGD retrovirus. Sequence alignment revealed that shRNA1 contained 7 nucleotide mismatches against mouse 6-PGD mRNA sequence. Western blot analysis confirmed the expression of mouse 6-PGD in infected cells; importantly, 6-PGD shRNA1 retained its ability to suppress endogenous 6-PGD expression upon doxycycline induction without affecting the expression of mouse 6-PGD. Overexpression of mouse 6-PGD alone enhanced H1975 cell growth. As expected, expression knockdown of endogenous human 6-PGD by shRNA1 again significantly blocked proliferation of H1975 cells. However, in the presence of exogenously expressed mouse 6-PGD, cell proliferation was significantly restored even when endogenous human 6-PGD was knocked down by shRNA1. Our result confirmed that the growth inhibition was due to 6-PGD knockdown and not a non-specific artifact of the shRNA. Our results strongly suggest that 6-PGD is indispensable in cell proliferation in NSCLC.

### Example 3

### Expression Knockdown of 6-PGD Induces Cellular Senescence and Upregulates p53 Expression

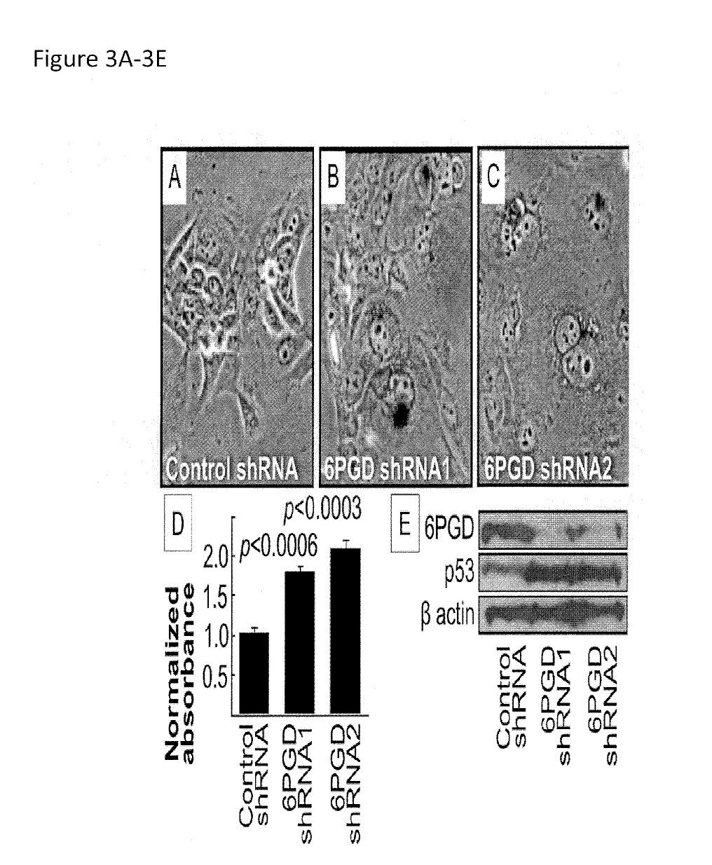

We noticed that the cells with 6-PGD knocked down were enlarged and flattened-shapes characteristic of cells undergoing senescence. We therefore stained the cells for senescence-associated beta galactosidase (SA-β-GAL) activity. Because shRNA1 and shRNA2 had the most dramatic effects on 6-PGD expression and cell proliferation, we opted to test these two shRNAs in these experiments. Indeed, 6-PGD knockdown by either shRNA1 or 2 significantly increased number of cells positive for SA-β-GAL staining (FIGS. 3A-C). Quantitative analysis using a chromogenic assay also showed that cells with 6-PGD knocked down had significantly higher SA-β-GAL activity in their lysates (FIG. 3D). Therefore, we concluded that suppression of 6-PGD induces cellular senescence in these cancer cells.

One of the main mechanisms for cellular senescence induction is the p53 pathway. Therefore we hypothesized that p53 expression might be increased in cells with 6-PGD knockdown. Indeed, knockdown of 6-PGD by either shRNA1 or shRNA2 significantly upregulated p53 expression in H1975 cells (FIG. 3E). Our results clearly demonstrate that expression knockdown of 6-PGD upregulates p53 protein level in H1975 cells and that this effect may be responsible for the onset of cellular senescence.

### Example 4

### Expression Knockdown of G6PD Only has Minor Effects on Cell Growth

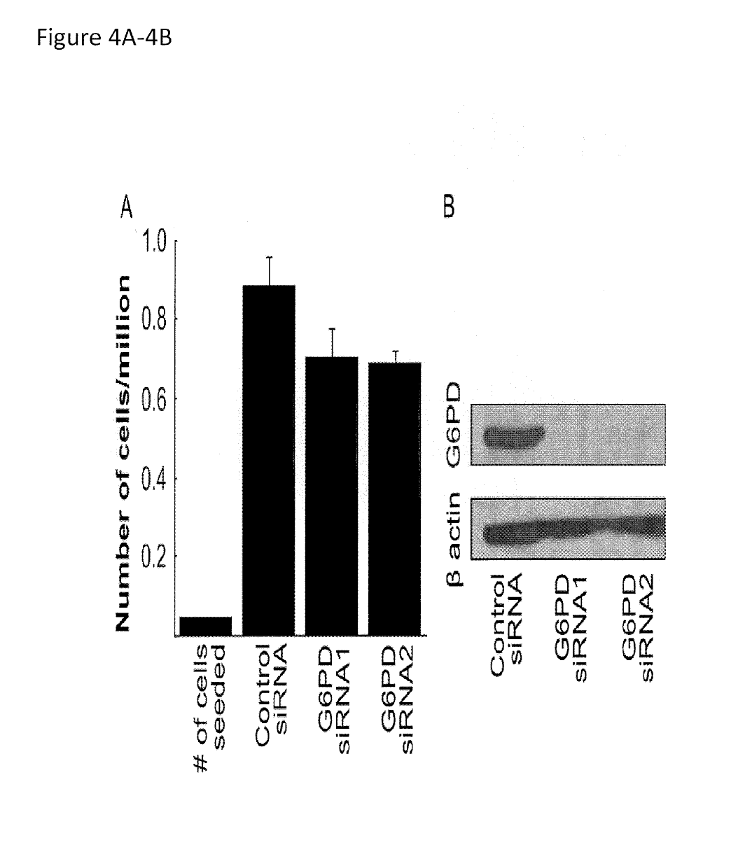

The dramatic decrease in cell proliferation when 6-PGD was knocked down implies that the oxidative branch of the pentose phosphate pathway (PPP) could be indispensable for cell growth. However, several published reports suggest that knockdown of G6PD, an enzyme upstream of 6-PGD, has only marginal effects in cell growth in vivo. To address this discrepancy, we next knocked down G6PD by transient siRNA transfection in H1975 cells. In contrast to 6-PGD knockdown, G6PD knockdown by two different siRNAs resulted in only minor inhibition of proliferation (FIG. 4). Therefore, the dramatic growth inhibition observed when 6-PGD was knocked down was not simply a result of PPP blockade.

### Example 5

### Expression Knockdown of 6-PGD does not Reduce Cellular NADPH Levels

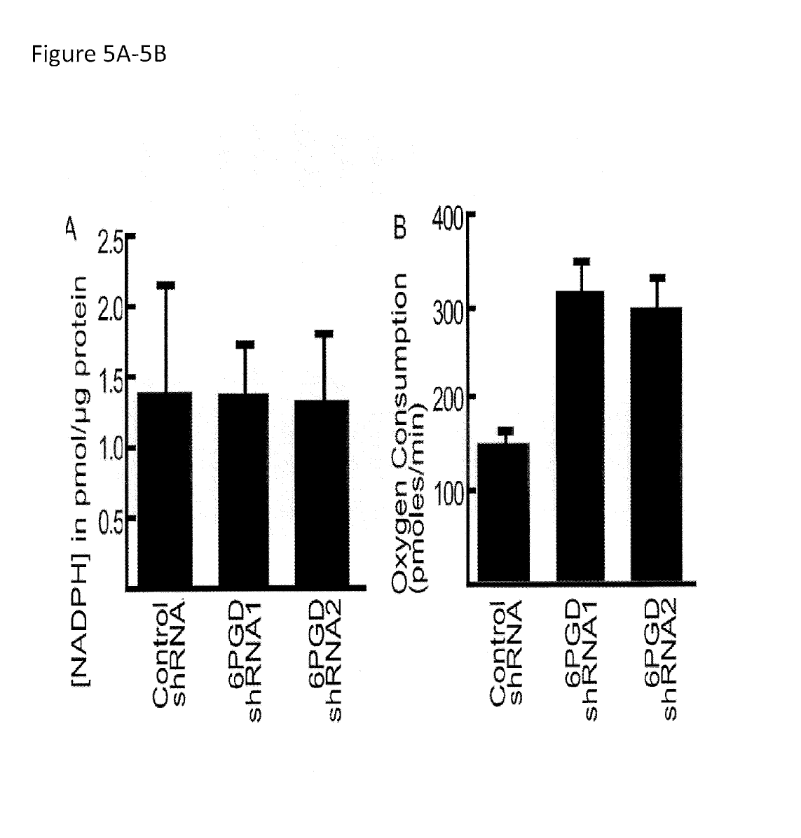

To further probe whether the growth inhibition caused by 6-PGD knockdown was due to a functional defect in the PPP, we examined cellular NADPH level before and after 6-PGD knockdown (FIG. 5A). Our results indicate that 6-PGD expression knockdown in H1975 did not cause a reduction in cellular NADPH level. Previously Filosa et al. also reported that shutting down PPP in mouse embryonic fibroblasts by means of G6PD gene deletion also resulted in unchanged levels of NADPH and glutathione. Therefore, our results suggest that 6-PGD affected proliferation in these cells in an NADPH-independent manner.

### Example 6

### Expression Knockdown of 6-PGD Upregulates Oxygen Consumption

The fact that 6-PGD expression knockdown does not affect NADPH level suggests that either PPP does not play a significant role in generation of NADPH or that there exists a compensatory pathway for NADPH production in these cells. We suspected that upon 6-PGD knockdown, these cells might have maintained NADPH level by enhanced glutaminolysis, another major mechanism for NADPH production. We therefore examined the rate of oxygen consumption of these cells, as NADPH production via glutaminolysis requires oxygen. Consistent with our expectation, knockdown of 6-PGD by either shRNA1 or 2 in H1975 caused a significant increase in oxygen consumption rate (OCR) (FIG. 5B). Our results show that knockdown of 6-PGD in H1975 causes an increase in oxygen consumption, supporting the notion that glutaminolysis is used to generate cellular NADPH.

### Example 7

### Changes in Levels of Metabolites Upon 6-PGD Knockdown

Since expression suppression of 6-PGD did not lower NADPH level, we were interested in determining whether the levels of other key cellular metabolites were affected by 6-PGD knockdown. Both control- and 6-PGD shRNA1-expressing H1975 cells were treated with doxycycline for three days. Metabolites were extracted for analysis by mass spectrometry. Results are grouped according to metabolic pathways (Table 5). Knockdown of 6-PGD resulted in a significant increase of metabolites in the oxidative branch of the PPP. Specifically, as might be expected, 6-phospho-D-gluconate and D-glucono-δ-lactone-6-phosphate levels were up by 11 and 7.9 fold, respectively. Direct determination of the effects in the non-oxidative branch was more difficult, as levels of some key intermediates such as xylulose-5-phosphate and D-sedo-heptulose-7-phosphate were not determined in our mass spectrometric analysis. However, there was no change in the level of total ribose phosphates, downstream products of the PPP. Importantly, levels of all the triphosphate nucleotides essential for DNA and RNA synthesis were essentially unchanged. Therefore, the activity of the non-oxidative branch of the PPP did not seem to have altered when 6-PGD was knocked down. Glycolysis also appeared to be affected by 6-PGD knockdown. Both glucose-6-phosphate and fructose-6-phosphate levels were increased, perhaps due to a backlog in the PPP. On the other hand, levels of fructose-1,6-bisphosphate, dihydroxyacetone phosphate, and glyceraldehyde-3-phosphate were all significantly lowered. We believe that these intermediates were shunted to the non-oxidative PPP via the transketolase reactions using glyceraldehyde-3-phosphate as the entry point. These transketolase reactions would compensate for the blockade at 6-PGD and provide a carbon source for ribose phosphate production and maintenance of dNTP/NTP levels. In contrast, the steady-state levels of metabolites in the TCA cycle did not change much when 6-PGD expression was suppressed. In addition, among all the species related to cellular REDOX that were measured in our analysis, only glutathione level appeared to be slightly lowered. In particular NADPH level was determined by this metabolomic analysis to be also unchanged by 6-PGD knockdown, consistent with result obtained by the colorimetric assay (FIG. 5A).

TABLE 5

| Sample Type | Fold Change | p value |
|---|---|---|
| PPP | | |
| D-Glucono-δ-lactone-6-phosphate | 7.9 | 0.001 |
| 6-Phospho-D-gluconate | 11.0 | 0.001 |
| Ribose-phosphate | 1.2 | 0.284 |
| Glycolysis | | |
| Glucose-6-phosphate | 1.8 | 0.006 |
| Fructose-6-phosphate | 1.4 | 0.005 |
| Fructose-1,6-bisphosphate | −4.6 | 0.000 |
| Dihyroxyacetone-phosphate | −5.9 | 0.047 |
| D-Glyceraldehdye-3-phosphate | −3.8 | 0.021 |
| 1,3-Diphopshateglycerate | 0.0 | 0.301 |
| 3-Phosphoglycerate | −2.1 | 0.001 |
| Phosphoenolpyruvate | −2.8 | 0.000 |
| Pyruvate | 1.3 | 0.392 |
| Lactate | −1.2 | 0.000 |
| NTPs/dNTPs | | |
| UTP | 1.2 | 0.100 |
| GTP | 1.5 | 0.168 |
| CTP | 1.0 | 0.905 |
| ATP | 1.2 | 0.052 |
| dTTP | −1.2 | 0.012 |
| dGTP | 1.2 | 0.125 |
| dCTP | 1.0 | 0.826 |
| dATP | −1.1 | 0.503 |
| TCA | | |
| Acetyl-CoA | 1.6 | 0.107 |
| Oxaloacetate | −1.1 | 0.267 |
| Citrate | 1.1 | 0.525 |
| Isocitrate | −1.1 | 0.464 |
| α-Ketoglutarate | −1.2 | 0.185 |
| Succinate | −1.3 | 0.052 |
| Fumarate | 1.0 | 0.905 |
| Malate | −1.3 | 0.017 |
| Glutamine/Glutamate | | |
| Glutamine | −1.1 | 0.187 |
| Glutamate | −1.1 | 0.479 |
| REDOX | | |
| NADPH | −1.1 | 0.852 |
| NADP+ | 1.3 | 0.094 |
| NADH | −1.5 | 0.171 |
| NAD+ | 1.2 | 0.407 |
| Glutathione disulfide | 1.0 | 0.977 |
| Glutathione | −1.3 | 0.044 |

### Example 8

### Glucose is Critical for the Inhibition of Proliferation of H1975 Cells when 6-PGD Expression is Suppressed

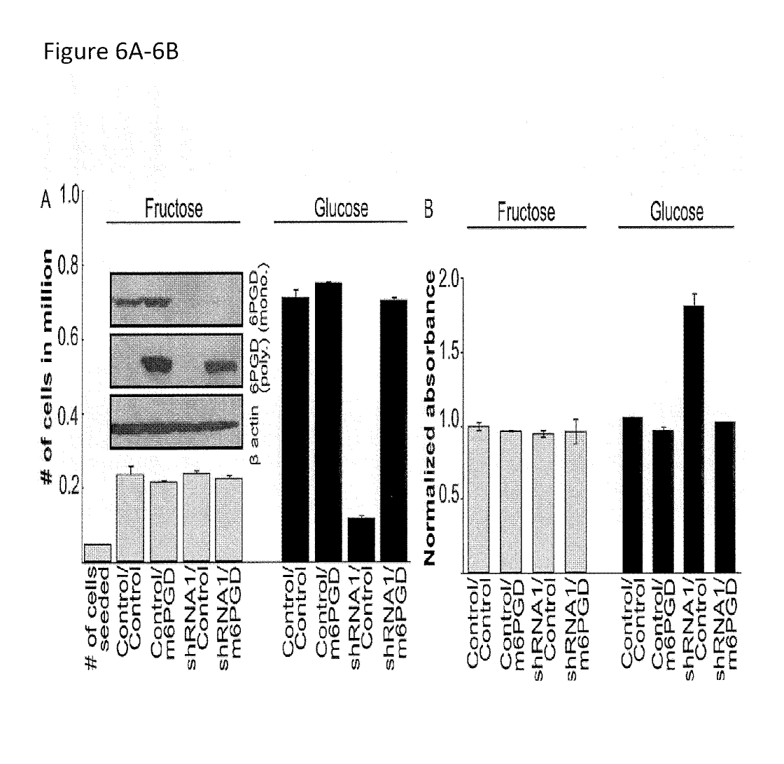

We tested whether the use of glucose or fructose in the medium might influence the phenotypic outcome of 6-PGD suppression. Fructose can be used to generate glycolytic metabolites via different pathways. For example, in the liver, fructose can be phosphorylated by fructokinase to yield fructose-1-phosphate and ultimately glyceraldehyde-3-phosphate to re-enter glycolysis. Fructose can also enter glycolysis when phosphorylated by hexokinase to become fructose-6-phosphate. We reasoned that if H1975 could metabolize fructose by these mechanisms, cells grown in fructose without glucose would likely be devoid of glucose-6-phosphate, bypassing the oxidative branch of the PPP. This scenario may be similar to the PPP shutdown by means of G6PD depletion. H1975 cells were treated with doxycycline to induce 6-PGD knockdown in 2 g/L fructose (no glucose). After four days of induction under this condition, endogenous 6-PGD was significantly knocked down. Then the cells were tested in proliferation assays in either 2 g/L fructose or glucose (FIG. 6A). The growth rate of H1975 in fructose was about a third of that in glucose. Therefore, H1975 cells could use fructose, although less efficiently than with glucose. As expected, in the presence of glucose, 6-PGD knockdown significantly inhibited proliferation, which was completely restored by exogenously expressed mouse 6-PGD. In contrast, in the presence of fructose, 6-PGD knockdown no longer inhibited cell growth when compared to the corresponding control. Similarly, relative senescence-associated β-GAL activity was only elevated in cells lacking 6-PGD grown in glucose but not fructose (FIG. 6B). These results are in agreement with the notion that the mechanism by which 6-PGD knockdown inhibited proliferation is separate from the fundamental functions of the oxidative branch of the PPP. Moreover, 6-PGD knockdown appeared to have “changed” the cells to respond to glucose as a growth inhibitor. Thus, 6-PGD can be thought of as a synthetically lethal target for these glycolytic tumor cells.

### Example 9

### G6PD Knockdown Rescues Proliferation of H1975 Cells Lacking 6-PGD

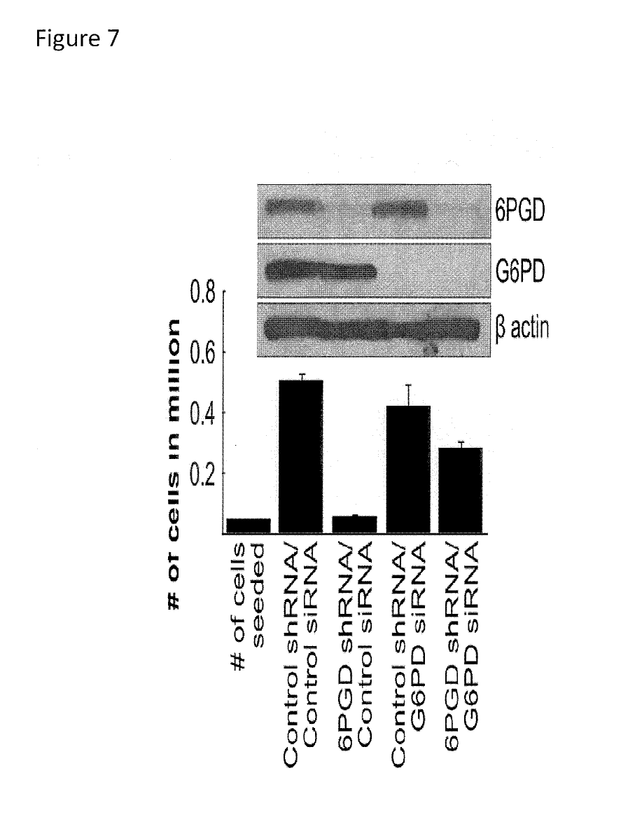

The fructose-rescue experiment led us to hypothesize that accumulation of oxidative PPP metabolites may be growth inhibitory. If true, knockdown of G6PD in cells lacking 6PGD should prevent accumulation of these metabolites and restore proliferation of H1975 cells even in the absence of 6-PGD. Indeed, transient knockdown of G6PD by siRNAs significantly rescued proliferation of cells with 6-PGD knocked down (FIG. 7). Thus, our results strongly suggest that 6-phospho-D-gluconate and D-glucono-δ-lactone-6-phosphate may be inhibitors of proliferation.

### Example 10

### 6-PGD Expression Knockdown Suppresses Tumor Growth in Mice

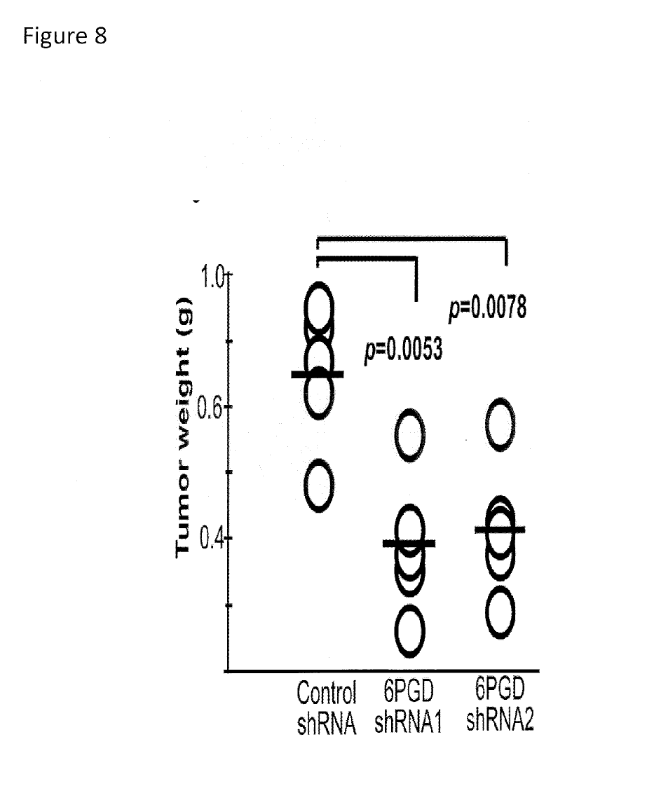

We sought to test whether 6-PGD knockdown would inhibit tumor growth in a xenograft model. Control shRNA-, 6-PGD shRNA1, and 6-PGD shRNA2-H1975 cells were induced with doxycycline in vitro for three days prior to subcutaneous injection into nude mice. Mice were fed with doxycycline supplemented in drinking water. Tumors were excised after 18 days and weighed. As shown in FIG. 8, knockdown of 6-PGD by either shRNA significantly retarded tumor growth.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known customary practice within the art to which the invention pertains and may be applied to the essential features herein before set forth.

All publications, patents, and patent applications are herein incorporated by reference in their entirety to the same extent as if each individual publication, patent or patent application was specifically and individually indicated to be incorporated by reference in its entirety.

What is claimed is:

1. A method of treating a subject having a proliferative disease, said method comprising administering to said subject a 6-phosphogluconate dehydrogenase antagonist in an amount sufficient to treat said proliferative disease.

2. A method of treating a subject having a proliferative disease, said method comprising administering to said subject a 6-phosphogluconate dehydrogenase antagonist in an amount sufficient to treat said proliferative disease, wherein said proliferative disease is resistant to gefitinib/erlotinib.

3. The method of claim 1 or 2, wherein said proliferative disease is lung cancer.

4. A method of treating a subject having lung cancer, said method comprising:
determining if said lung cancer is resistant to gefitinib/erlotinib, and
administering to said subject determined to have a lung cancer resistant to gefitinib/erlotinib a 6-phosphogluconate dehydrogenase antagonist in an amount sufficient to treat said lung cancer.

5. The method of claims 1-4, wherein said 6-phosphogluconate dehydrogenase antagonist is an RNAi agent, an anti-6-PGD antibody, or glucose 1,6-diphosphate.

6. The method of claims 1-4, wherein said lung cancer is selected from a group consisting of non-small cell lung cancer, small-cell lung cancer, carcinoid, sarcoma, squamous cell cancer, adenocarcinoma, and large cell carcinoma.

7. The method of claims 1-6, wherein said 6-phosphogluconate dehydrogenase antagonist is administered with an anticancer agent.

8. A method for identifying a compound for treating a proliferative disease, said method comprising contacting a cell with a candidate compound and measuring 6-phosphogluconate dehydrogenase activity, wherein the presence of a decreased level of 6-phosphogluconate dehydrogenase activity in said cell, as compared to a normal reference sample, identifies the compound as a treatment for a proliferative disease.

9. A method for identifying a treatment for proliferative disease, said method comprising contacting a cell with gefitinib/erlotinib and a candidate compound and measuring 6-phosphogluconate dehydrogenase activity, wherein the presence of a decreased level of 6-phosphogluconate dehydrogenase activity in said cell, as compared to a normal reference sample, identifies the compound as a treatment for a proliferative disease resistant to gefitinib/erlotinib.

10. The method of claim 8 or 9 wherein said proliferative disease is lung cancer.

11. The method of claim 8 or 9 wherein said cell is derived from a patient with lung cancer.

12. The method of claim 11 wherein said cell is resistant to gefitinib/erlotinib.

13. The method of claim 8 or 9, wherein said proliferative disease is selected from the group consisting of leukemia, brain cancer, bladder cancer, breast cancer, cervical cancer, colorectal cancer, endometrial cancer, esophageal cancer, head and neck cancer, liver cancer, lung cancer, lymphoma, ovarian cancer, pancreatic cancer, prostate cancer, renal cancer, skin cancer, stomach cancer, testis cancer, thyroid cancer, and urothelial cancer.

14. The method of claim 10, wherein said proliferative disease is lung cancer and said lung cancer is selected from the group consisting of non-small cell lung cancer, small-cell lung cancer, carcinoid, sarcoma, squamous cell cancer, adenocarcinoma, and large cell carcinoma.

15. A method for diagnosing a subject as having, or having a predisposition to a proliferative disease, said method comprising:
determining the level of 6-PGD activity in a sample from said subject,
comparing said level of 6-PGD activity with a normal reference sample, wherein the presence of an increased level of 6-PGD activity, as compared to said normal reference sample, results in diagnosing said subject as having, or having a predisposition to, said proliferative disease and,
administering to said subject a 6-PGD antagonist in an amount sufficient to treat said proliferative disease.

16. The method of claim 15, wherein said antagonist is an RNAi agent or an anti-6-PGD antibody.

17. The method of claim 15, wherein said proliferative disease is selected from the group consisting of leukemia, brain cancer, bladder cancer, breast cancer, cervical cancer, colorectal cancer, endometrial cancer, esophageal cancer, head and neck cancer, liver cancer, lung cancer, lymphoma, ovarian cancer, pancreatic cancer, prostate cancer, renal cancer, skin cancer, stomach cancer, testis cancer, thyroid cancer, and urothelial cancer.

18. The method of claim 15, wherein said proliferative disease is lung cancer and said lung cancer is selected from the group consisting of non-small cell lung cancer, small-cell lung cancer, carcinoid, sarcoma, squamous cell cancer, adenocarcinoma, and large cell carcinoma.

19. A composition comprising a 6-PGD antagonist and an anticancer agent.

20. The composition of claim 19, wherein said anticancer agent is one or more of a chemotherapeutic agent, an antiangiogenic agent, an immunomodulatory agent, or an agent for metabolic therapy.
